(19) United States
(12) Patent Application Publication
(10) Pub. No.: US 2006/0235284 A1
(43) Pub. Date: Oct. 19, 2006

(54) Glucose extraction patch and its manufacturing process

(76) Inventor: Gi-Ja Lee, Kyonggi-do (KR)

Correspondence Address: MORGAN & FINNEGAN, L.L.P., 3 WORLD FINANCIAL CENTER, NEW YORK, NY, 10281-2101, US

(21) Appl. No.: 10/526,112

(22) PCT Filed: Aug. 30, 2002
(86) PCT No.: PCT/KR02/01634

§ 371 Date: Feb. 16, 2006

(51) Int. Cl. A61B 5/05

(52) U.S. Cl. 600/345; 600/362; 600/365

(57) ABSTRACT

Glucose extraction patch and its manufacturing process are provided. The patch comprises two gel discs containing enzymes that react with glucose to produce hydrogen peroxide, a frame for having two holes for accommodating the discs, a supporter for holding the discs, and a flexible printed circuit. The printed circuit includes electrodes for contacts with the gel discs, terminals for electrical connection with a measuring device, and circuits for electrical connection between the electrodes and the terminals. The electrodes are formed on the printed circuit by silk screening. Preprocessing may be applied to the electrodes after they are formed on the printed circuit.

### TECHNICAL FIELD

The present invention relates to a glucose extraction patch and its manufacturing process, and in particular, to a glucose extraction patch that can easily be manufactured and provides convenience in use, as well as a manufacturing process thereof.

### BACKGROUND ART

A conventional blood glucose measuring instrument measures blood sugar level by absorbing blood sample to a sensor with enzymes fixed on the surface of its electrodes using high polymer, after a blood sample has been taken from a finger by wounding the finger using a needle. However, since such invasion type glucose measuring instrument causes pain and fear for a patient, some of the patients requiring periodic monitoring of their glucose level such as diabetics even refuse using it due to its discomfort. Further, a conventional invasion type glucose measuring instrument fails to continuously monitor glucose level, although there are often occasions when the glucose level of a patient rises to dangerous ranges instantly, requiring an immediate treatment.

Under these circumstances, diverse methods allowing measuring of concentrations of a substance in blood without taking a blood sample have been developed. For example, U.S. Pat. No. 5,267,152 by Yang et al. discloses a non-invasive measurement method for measuring blood glucose concentrations using near-infrared radiation diffusion-reflection laser spectroscopy. Similar near-infrared spectroscopy instruments are described in U.S. Pat. No. 5,086,229 by Rosenthal et al. and in U.S. Pat. No. 4,975,581 by Robinson et al. However, these methods have not yet been put to practical use due to their technical problems.

Further, U.S. Pat. No. 5,139,023 by Stanley et al. describes an epithelial blood glucose monitoring apparatus using a glucose permeation enhancer (e.g., bile acid salt) capable of increasing the glucose permeability across an epithelial membrane by the glucose concentration gradient established between the interstitial fluid and the glucose receiving medium. And U.S. Pat. No. 5,036,861 by Sembrowich et al. describes a passive glucose monitoring method, wherein sample of pharmacologically induced sweat is collected using a patch containing cholinergic agent for inducing a localized sweating response from an exocrine gland. Similar sweat collecting apparatus are described in U.S. Pat. No. 6,076,273 by Schoendorfer et al. and in U.S. Pat. No. 5,140,985 by Schroeder et al.

Also, U.S. Pat. No. 5,279,543 by Glickfield et al. describes a device for non-invasive sampling (or delivery) of substances to a reservoir on a membrane of the mammal using iontophoresis (electric osmosis). Glickfield et al. further suggest that this sampling method can be combined with a glucose specific biosensor or a glucose specific electrode for the purpose of blood glucose monitoring. Furthermore, International Publication WO 96/00110 by Tamada et al. describes an iontophoretic device for epithelial monitoring of a target substance, wherein an iontophoretic electrode is used for transmitting a sample to a reservoir and a biosensor is used for detecting a target sample in the reservoir.

However, these apparatus or devices have the drawbacks that they fail to provide a construction easily usable by patients and that they do not allow a continuous monitoring of the blood glucose level.

Although Korean Patent Publication No. 1997-7003790 discloses, in view of the above, a device using two patches, this invention has the same problem that it does not allow a patient to perform his activities freely with such patches attached on him. In order to solve this problem, Korean Patent Publication No. 1999-0077833 provides a device with just one patch. However, the problem with this device is that its construction is so complicated that it is not easily assembled and is of high cost. In addition, the measuring results obtained from such device are insufficient for use in the practice.

### DISCLOSURE OF THE INVENTION

The present invention, conceived to solve the above problems, aims to provide a glucose extraction patch having a simple construction to allow a patient to perform his activities freely, inclusive of the electrodes used therein, and a process for manufacturing the same.

Another objective of the present invention is to provide a glucose extraction patch and electrodes used in the patch that can easily be manufactured with low costs, and its manufacturing process.

Still another objective of the present invention is to provide an electrode composition that can enhance the measuring accuracy, and a method for preprocessing of the same.

### BRIEF DESCRIPTION OF THE DRAWING

FIGS. 1a and 1b illustrate a glucose monitoring device in accordance with the present invention.

FIG. 2a is an exploded perspective view showing construction of a patch in accordance with one embodiment of the present invention.

FIG. 2b is an exploded perspective view showing construction of a patch in accordance with another embodiment of the present invention.

FIG. 2c is a cross-sectional view showing the frame of a patch in accordance with still another embodiment of the present invention.

FIG. 3 is an exploded perspective view showing construction of the printed circuits.

FIG. 4 shows drawings illustrating a process wherein electrodes are formed by silk-screen method.

FIGS. 5a through 5c are drawings showing various forms of electrodes constructions.

### BEST MODE FOR CARRYING OUT THE INVENTION

A glucose extraction patch in accordance with the present invention comprises two hydro gel discs each of which containing enzyme for generating hydrogen peroxide in reaction with glucose, a frame with two holes formed in it for accommodation of the two gel discs, and a flexible circuit board attached on the upper part of the frame having electrodes formed at positions facing the two gel discs respectively, terminals for connection with a measuring instrument, and a circuit means for electric connections between the electrodes and the terminals.

Further, a film with two holes formed in it, the holes having a smaller diameter than those of the frame and being formed to face the holes of the frame, can be attached on the bottom of the frame for support of the gel discs. Alternatively, supporting wings slightly extruding to the center of the holes from the bottom part of the holes in the frame can be formed for support of the gel discs.

The first extraction electrode, having a ring shape, is installed at a location facing one of the hydro gel discs; the second extraction electrode, having a broken ring shape, is installed at a location facing the other hydro gel disc; the circle shape working electrode is installed at a location inside of the broken ring shape second extraction electrode; and the reference electrode as well as the counter electrode are connected in series at location where the ring of the second extraction electrode is broken. Or, these electrodes can be connected in parallel at the same location.

Alternatively, the first extraction electrode, having a ring shape, is installed at a location facing one of the hydro gel discs; the second extraction electrode, having also a ring shape, is installed at a location facing the other hydro gel disc; the circle shape working electrode is installed at a location inside of the ring shape second extraction electrode; and the reference electrode as well as the counter electrode, each having a half ring shape, are located to surround the outside of the ring shape second extraction electrode.

Here, it is preferable that the electrodes, the terminals and the circuits are formed on the same surface of a flexible circuit board and the circuit board parts where the terminals are formed are cut-off to allow the terminals to be exposed to the rear surface of the circuit board.

The above patch is manufactured through a process comprising: the first step wherein the hydro gel discs are put into holes in the frame provided for accommodation thereof; the second step wherein the terminals as wells as the circuit patterns are formed with copper wire on the flexible circuit board film; the third step wherein a mask with an electrode pattern made of a first material is set on the surface where the above circuit patterns are formed, and ink made of the first material is sprayed thereon and then cured; the fourth step wherein the above third step is repeated for each electrode material; the fifth step wherein the film with electrodes formed on it is cured for a predetermined period of time, and the sixth step wherein the above cured film is sprayed with adhesive except for the electrode parts, and then adhered to the frame containing the hydro gel discs.

If the above electrodes undergo, after the above fifth step, an additional step of oxidation using cyclic voltammetry in the range of 0.0˜1.2V, after they have been dipped in a sulfuric acid solution of 0.01˜10M, the electrodes show an enhanced performance. Furthermore, the curing time in the above fifth step shall preferably be three hours.

A still further step can be added in which a film having holes with a diameter smaller than that of the above hydro gel discs is adhered to the surface of the frame opposite to the surface on which the flexible circuit board is attached, in a manner that the holes correspond to the above hydro gel discs.

A detailed description of the preferred embodiments of the present invention follows below making reference to the attached drawings.

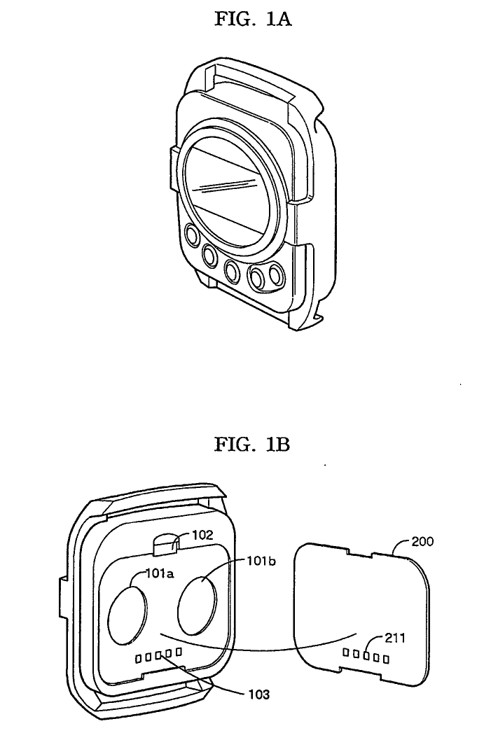

A patch in accordance with the present invention is used in a glucose monitoring device. Such glucose monitoring device can have a wrist watch form as shown in FIG. 1a, comprising on its front side an LCD for displaying the measured glucose value as well as buttons for operating the device.

On the rear side of the device, a disposable patch 200 can be affixed as shown in FIG. 1b. The device is equipped with a locker 102 for this end, and further comprises spaces (101a, 101b) for accommodation of batteries.

If the device is tightened to a patient's wrist, a direct contact is established between the rear surface of the patch and the patient's skin. Upon application of current to the patch 200 from the device, glucose is extracted from the patient's skin to the patch by electro osmotic pressure. The extracted glucose triggers an electrochemical reaction to the hydro gel in the patch, which reaction generates an electric current. The device can monitor blood glucose level by measuring the current thus generated.

The patch 200 is equipped with terminals 211 to enable electric connections. If the patch 200 is installed in the device, electric connections between the terminals 211 and the terminals 103 on the rear side of the device are established. The device not only applies current, but also measures the current generated by the electro chemical reaction through these terminals.

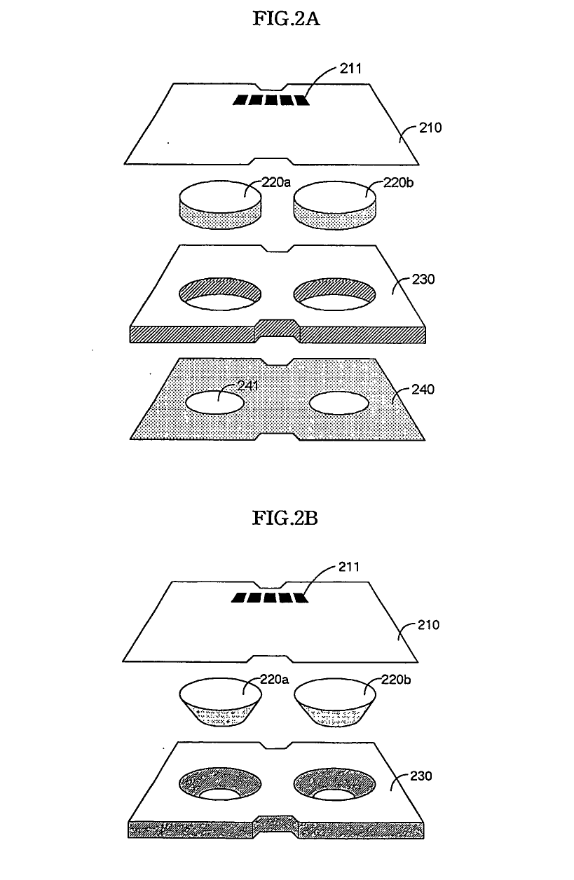

As next, a detailed description on the construction of the patch follows, making reference to FIGS. 2a and 2b.

FIG. 2a is an exploded perspective view showing construction of a patch in accordance with one embodiment of the present invention.

The patch 200 comprises two gel discs 220a, 220b, a frame 230 for accommodation of the two gel discs, a film 240 for support of the gel discs, and printed circuits 210, which comprise electrodes for contacts with the gel discs 220a, 220b. The electrodes are connected to the terminals 211 with conductive circuits such as copper wire.

The gel discs 220a, 220b are made of hydro gel comprising enzyme. U.S. Pat. No. 5,139,023, Korean Patent Publication No. 1999-028884, Korean Patent Publication No. 1999-0077833, etc. contain descriptions in this respect, but the present invention is not limited to a specific gel composition.

The frame 230 comprises holes having a size same as the gel discs 220a, 220b in order to accommodate the gel discs. Any material that is harmless to human such as Teflon, and the like can be used for the frame 230.

The film 240 supports the gel discs 220a, 220b in a manner that they are not separated from the frame 230, while it contains holes 241 in it allowing direct contact of the gel discs 220a, 220b with the skin. The size of the holes 241 shall be smaller than that of the gel discs 220a, 220b to ensure that the gel discs 220a, 220b remain in the frame 230.

FIG. 1b illustrates another embodiment wherein no film 240 is required. In FIG. 2b, a separation of the gel discs 220a, 220b from the frame 230 is prevented by shaping the gel discs 220a, 220b in such a manner that the upper part thereof is larger than the bottom part thereof and shaping the holes in the frame 230 correspondingly.

Alternatively, it is also possible that the gel discs 220a, 220b have the same shape as in FIG. 2a, but the holes in the frame 230 are differently shaped as illustrated in FIG. 2c, i.e., a wing part 231 is added at the bottom part of the holes, so that a separation of the gel discs 220a, 220b from the frame 230 is prevented without using a film. Here, the wing part 231 shall be sufficiently thin to allow direct contact of the gel discs 220a, 220b to the skin.

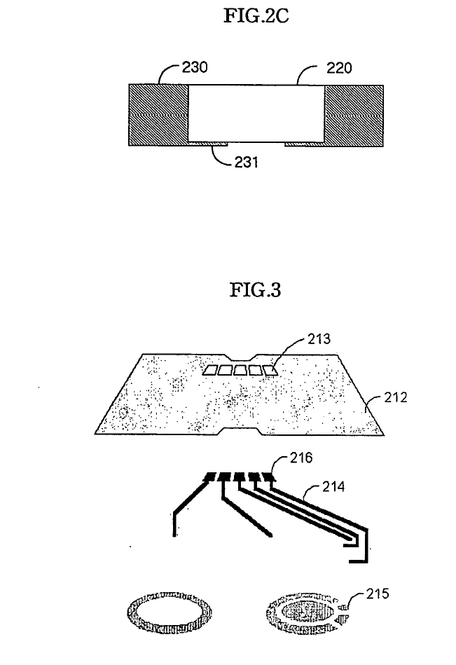

The printed circuits 210 are consisted of a polyimid film 212, copper wire 214, and electrodes 215, as shown in the exploded perspective view illustrated in FIG. 3.

Any material suitable for use as a flexible printed circuit board can be used for the film 212. The film 212 is provided with holes 213, to allow the electrodes 216 made of copper wire be exposed over the patch 200. The copper wire 214 can be formed on the surface of the film 212 through a general process for manufacturing a flexible circuit board.

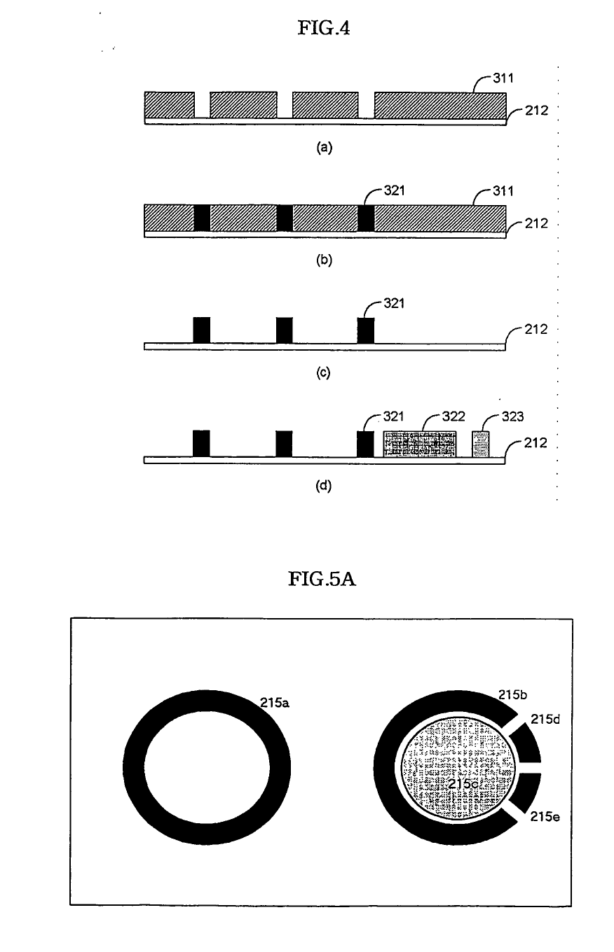

Then, the electrodes 215 are generated on the film 212 with copper wire 214 formed on it. The electrodes 215 can be generated on the film through a silkscreen process. FIG. 4 shows drawings illustrating a process wherein electrodes are formed by silkscreen method.

The electrodes can be made of different material in accordance with their purposes. At first, a mask 311 with shapes of electrodes having one material is put on the film 212 (step a). Then, ink 321 for this material is sprayed on the mask 311 (step b). Subsequently, the film 212 is cured at a high temperature, e.g. at 130° C., after the mask 311 has been removed (step c). Here, the curing conditions are similar to the curing conditions between a first and a second color printing in a general silkscreen process. Although experiments have shown a ten minutes curing at 130° C. to be sufficient, the present invention is not limited thereto.

After such curing, the above steps (a) through (c) are repeated for each of other materials. If repetition of the above steps for each material has been completed, a final curing of three hours at 130° C. is performed. As a result, a film 212, as illustrated in FIG. 4, with electrodes made of inks with different materials (321, 322, 323) formed on it is generated.

Table 1 below shows current changes by glucose based on the final curing time. As shown in the Table, the maximum current change has occurred at a curing time of three hours. This means that a most sensitive reaction can be obtained at a curing time of three hours.

TABLE 1

| Curing Time | Current Change (nA) by Glucose |
|---|---|
| 1 h | 44.3 |
| 2 h | 41.1 |
| 3 h | 56.7 |
| 4 h | 49.7 |
| 5 h | 46.3 |

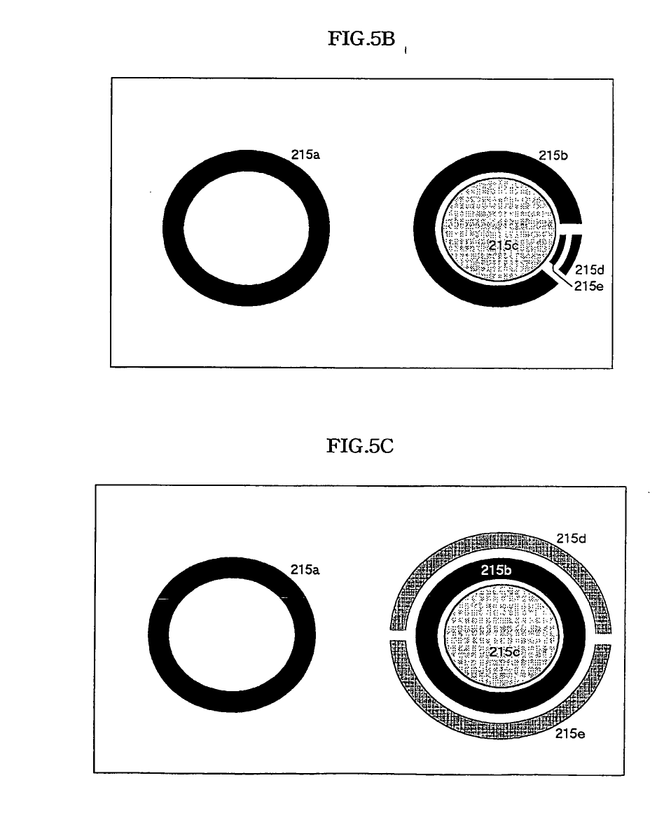

FIGS. 5a through 5c are drawings showing various forms of electrodes constructions. A total of five electrodes are formed in the patch 200, i.e. two extraction electrodes 215a, 215b, a working electrode 215c, a reference electrode 215d, and a counter electrode 215e.

The extraction electrodes 215a, 215b, being electrodes for extraction of glucose from skin, are formed using platinum ink containing carbon, or ink containing Ag/AgCl.

The working electrode 215c, being an electrode that applies voltage for measuring the current generated by reaction of the hydro gel with glucose extracted, is formed using ink containing platinum and carbon in an appropriate ratio. Table 2 below shows current changes based on the ratio by weight of platinum and carbon. The electrodes manufactured for the above experiment have been preprocessed as described far below.

TABLE 2

| | | | |
|---|---|---|---|
| | Current change | | |
| Electrode Ratio | (nA) | Noise | S/N |

| | | | | |
|---|---|---|---|---|
| Pt | (plate) | 80.6 | 3.0 | 26.86 |
| Pt | (ink) | 150.9 | 3.6 | 41.91 |
| Pt/C | (95/5) | 156.2 | 1.9 | 82.21 |
| Pt/C | (86/14) | 103 | 1.3 | 79.23 |
| Pt/C | (70/30) | 90.3 | 1.2 | 75.25 |
| Pt/C | (50/50) | 56.7 | 0.9 | 63 |
| Pt/C | (30/70) | 25 | 0.5 | 50 |

As shown in the above Table, the most sensitive reaction was obtained when the ratio by weight of platinum to carbon was 95:5.

The reference electrode 215d, being formed using ink containing Ag/AgCl, serves as the basic electric potential in measuring the current generated by reaction of the hydro gel and glucose extracted.

The counter electrode 215e, being formed using platinum ink containing carbon, measures the current generated by reaction of the hydro gel and glucose extracted.

Here, the electrodes show an improved performance when they have undergone a preprocessing procedure. The above preprocessing is carried out in a manner that the electrodes are put into an oxidation procedure in a range of 0.0˜1.2V using cyclic voltammetry after a certain part of these electrodes have been dipped into a 0.01˜10M sulfuric acid solution.

Table 3 below shows current changes in the electrodes with or without a preprocessing with 0.5M sulfuric acid solution. Here, predetermined parts of the strip form electrodes have been dipped into 3 ml physiological salt solution containing a predetermined amount of glucose oxidation enzyme in a 38° C. thermostatic cell. Subsequently, changes in the current generated by reactions of glucose with 500 μM concentration with the glucose oxidation enzyme have been measured while a voltage of 0.4V is applied using a potentiostat to the above preprocessed electrodes.

TABLE 3

| Ratio of Pt in | | Current | | |
|---|---|---|---|---|
| Electrodes (wt %) | Preprocessing | Change (nA) | Noise | S/N |
| 100 | No | 27.8 | 1.0 | 27.8 |
| | Yes | 87.2 | 1.5 | 58.1 |
| 95 | No | 9.54 | 1.0 | 9.54 |
| | Yes | 86.9 | 0.8 | 108 |
| 70 | No | 5.35 | 0.5 | 10.7 |
| | Yes | 18.5 | 0.6 | 30.8 |
| 50 | No | 8.98 | 0.5 | 18.0 |
| | Yes | 30.5 | 0.7 | 43.6 |

As shown in the above Table, preprocessed electrodes with a Pt ratio of 95% by weight have yielded the best performance, and in general, electrodes with preprocessing could more effectively sense the current changes by glucose than the electrodes without a preprocessing.

A flexible circuit board 210 with such electrodes formed on it is then adhered to the frame 230 using an adhesion means, etc. Here, the adhesive is sprayed on the circuit board 210 except for the parts where the electrodes are formed, i.e. the parts where the electrodes contact the hydro gels.

Although the present invention has been described above with reference to the embodiment examples and the drawings, the scope of rights of the present invention is not limited thereto, but rather, shall be determined by the claims attached herein after and their equivalents, allowing various modifications and adaptations without departing the spirit of the present invention, as those skilled in the art will understand.

### INDUSTRIAL APPLICABILITY

As described above, the present invention provides a user with convenience in use by constructing the electrodes and the hydro gel discs in one glucose extraction patch to enable easy installment in/detachment from the glucose measurement apparatus.

Further, the present invention provides an easy manufacturing process of a glucose extraction patch utilizing silkscreen method and printed circuit board method in manufacturing the electrodes, terminals, and circuits of the above patch.

In addition, the present invention can enhance the measuring performance by using material of specific composition for the electrodes and by providing a specific preprocessing to the electrodes.

1. A glucose extraction patch comprising:
two hydro gel discs each of which containing enzyme for generating hydrogen peroxide in reaction with glucose;
a frame with two holes formed in it for accommodation of said two gel discs; and
a flexible circuit board attached on the upper part of said frame having electrodes formed at positions facing said two gel discs respectively, terminals for connection with a measuring instrument, and a circuit means for electric connections between said electrodes and said terminals.

2. The glucose extraction patch of claim 1, comprising additionally a film attached on the bottom of said frame with two holes formed in it, said holes having a smaller diameter than those of said frame and being formed to face said holes of said frame.

3. The glucose extraction patch of claim 1, comprising additionally supporting wings formed slightly extruding to the center of said holes from the bottom part of said holes in said frame for support of said gel discs.

4. The glucose extraction patch of claim 1, wherein the bottoms of said gel discs are smaller sized than the upper surfaces thereof, and said two holes of said frame are shaped to accommodate said gel discs.

5. A glucose extraction patch in any one of claims 1 through 4, wherein said electrodes comprise:
a first and a second extraction electrodes formed using platinum ink containing carbon, or ink containing Ag/AgCl, which extract glucose from an intact skin;
a working electrode formed using ink containing platinum and carbon, which applies voltage for measuring the current generated by reaction of said hydro gel with glucose extracted;
a reference electrode formed using ink containing Ag/AgCl, which serves as the basic electric potential in measuring said current generated by reaction of said hydro gel and glucose extracted; and
a counter electrode formed using platinum ink containing carbon, which measures said current generated by reaction of said hydro gel and glucose extracted.

6. The glucose extraction patch of claim 5, wherein the ratio of platinum to carbon in said working electrode is 95:5 by weight.

7. The glucose extraction patch of claim 5, wherein
said first extraction electrode, having a ring shape, is installed at a location facing one of said hydro gel discs;
said second extraction electrode, having a broken ring shape, is installed at a location facing the other hydro gel disc;
said working electrode, having a circle shape, is installed at a location inside of said broken ring shape second extraction electrode; and
said reference electrode as well as said counter electrode are connected in series at location where said ring of said second extraction electrode is broken.

8. The glucose extraction patch of claim 5, wherein
said first extraction electrode, having a ring shape, is installed at a location facing one of said hydro gel discs;
said second extraction electrode, having a broken ring shape, is installed at a location facing the other hydro gel disc;
said working electrode, having a circle shape, is installed at a location inside of said broken ring shape second extraction electrode; and
said reference electrode as well as said counter electrode are connected in parallel at location where said ring of said second extraction electrode is broken.

9. The glucose extraction patch of claim 5, wherein
said first extraction electrode, having a ring shape, is installed at a location facing one of said hydro gel discs;
said second extraction electrode, having also a ring shape, is installed at a location facing the other hydro gel disc;
said working electrode, having a circle shape, is installed at a location inside of said ring shape second extraction electrode; and
said reference electrode as well as said counter electrode, each having a half ring shape, are located to surround the outside of said ring shape second extraction electrode.

10. The glucose extraction patch of claim 5, wherein
said electrodes undergo an additional preprocessing step of oxidation using cyclic voltammetry in the range of 0.0˜1.2V, after they have been dipped in a sulfuric acid solution of 0.01˜10M.

11. The glucose extraction patch of claim 1, wherein said electrodes, said terminals and said circuits are formed on the same surface of a flexible circuit board and the circuit board parts where said terminals are formed are cut-off to allow said terminals to be exposed to the rear surface of said circuit board.

12. The glucose extraction patch of claim 11, wherein said flexible circuit board is adhered to said frame using an insulating adhesion means, whereby said adhesive is sprayed on said circuit board except for the parts of said electrodes.

13. A glucose extraction patch manufacturing process comprising:
a first step wherein said hydro gel discs are put into holes in said frame provided for accommodation thereof;
a second step wherein said terminals as wells as said circuit patterns are formed with copper wire on flexible circuit board film;
a third step wherein a mask with an electrode pattern made of a first material is set on the surface where said circuit patterns are formed, and ink made of said first material is sprayed thereon and then cured;
a fourth step wherein said third step is repeated for each electrode material;
a fifth step wherein said film with electrodes formed on it is cured for a predetermined period of time; and
a sixth step wherein said cured film is sprayed with adhesive except for the electrode parts, and then adhered to said frame containing said hydro gel discs.

14. The glucose extraction patch manufacturing process of claim 13, wherein said electrodes undergo, after said fifth step, an additional step of oxidation using cyclic voltammetry in the range of 0.0˜1.2V, after they have been dipped in a sulfuric acid solution of 0.01˜10M.

15. The glucose extraction patch manufacturing process of claim 13, wherein said curing time at said fifth step is three hours.

16. The glucose extraction patch manufacturing process of claim 13, wherein the parts of said flexible circuit board film corresponding to said terminals are all cut-off.

17. The glucose extraction patch manufacturing process of claim 13 comprising an additional step wherein a film having holes with a diameter smaller than that of said hydro gel discs is adhered to the surface of said frame opposite to the surface on which said flexible circuit board is attached, in a manner that said holes correspond to said hydro gel discs.
